(19) United States
(12) Patent Application Publication
(10) Pub. No.: US 2023/0181115 A1
(43) Pub. Date: Jun. 15, 2023

(54) METHOD AND SYSTEM TO DETECT A FIRST GAS IN THE SURROUNDING OF A MATTRESS ASSEMBLY

(71) Applicant: PHILIP MORRIS PRODUCTS S.A., Neuchâtel (CH)

(72) Inventors: Ricardo CALI, Mannheim (DE); Tobias LIPP, Santiago de Compostela (ES); Toney Moses RAJAN, Mannheim (DE)

(73) Assignee: PHILIP MORRIS PRODUCTS S.A., Neuchâtel (CH)

(21) Appl. No.: 17/923,695

(22) PCT Filed: May 4, 2021
(86) PCT No.: PCT/EP2021/061737

§ 371 Date: Nov. 7, 2022

(30) Foreign Application Priority Data
May 7, 2020 (EP) 20173546.1

(51) Int. Cl. A61B 5/00; A61B 5/083; A61B 5/103

(52) U.S. Cl. CPC A61B 5/6891; A61B 5/0836; A61B 5/1036

(57) ABSTRACT

The invention relates to a system to detect a first gas, the system comprising: a mattress assembly defining a support surface including: o a first array of gas sensors, each gas sensor of the first array being adapted to measure an amount of a first gas in the surrounding of a portion of the support surface and adapted to output data indicative of the measured amount of the first gas, so that the amount of the first gas in a first array of surface portions of the support surface is sensed over time; and o a transmitter adapted to send a signal to an external device containing data indicative of the measured amount of the first gas. The invention relates also to a method to detect the first gas.

The present invention relates to a method and a system to detect a first gas in the surrounding of a mattress assembly, where the method and the system may detect the gas concentrations in different locations of a support surface defined by the mattress assembly.

Monitoring the health status of sleepers can be important due to incidences related to respiratory or breathing problems. Furthermore, monitoring the health status of sleepers can be important due to other health issues, like gastro-intestinal problems or infectious disease with symptoms such as fever.

Systems and methods are known in the art to monitor the health of persons while sleeping. These systems use various sensors, for example cameras, infrared sources or temperature and humidity sensors for monitoring given parameters. Using such sensors allows to monitor certain patterns, like body movements, body temperature or sleeping positions. Others can be informed about the results of the monitoring by a connected alerting device. Nevertheless, many known systems are not very precise due to a single type of analysis. Furthermore, often wearable sensors, like bracelets, placed close to a sleeping body may generate misleading monitoring results due to the person's movements.

Therefore, it would be desirable to render available a reliable monitoring system and method which could monitor environment of a body accurately, so that for example the health state of a sleeper can be monitored during their sleeping. Furthermore, it would be desirable to minimise the risk of performing an incorrect installation of these monitoring sensors on beds or mattresses.

According to an aspect, the invention relates to a system to detect a first gas, the system comprising a mattress assembly defining a support surface. The mattress assembly may include a first array of gas sensors, each gas sensor of the first array being adapted to measure an amount of a first gas in the surrounding of a portion of the support surface and adapted to output data indicative of the measured amount of the first gas, so that the amount of the first gas in a first array of surface portions of the support surface is sensed over time. The mattress assembly may include a transmitter adapted to send a signal to an external device containing data indicative of the measured amount of the first gas.

In the method of the invention, more than one gas sensor is used, so that the concentration of a first gas can be measured in the surrounding of more than one location of the support surface. In this way, if a body is located on the support surface, although a measurement of a gas sensor can be defective, the other measurements may still give accurate results. Furthermore, measuring the concentration of the first gas in more than a location may allow to determine the position of various body parts, for example of the head of a body, due for example to a first gas consumption or release higher than in other locations. Also, when recorded over time, a breathing cycle of the sleeper may be established from the local variation of concentration of specific gases, like for example oxygen or carbon dioxide.

The system of the invention includes a mattress assembly. The mattress assembly may be a single element. In this case, all components of the system are an integral part of a mattress. The mattress assembly on the other hand may include more than a single element. For example, the mattress may include a mattress and a mattress topper, separated from the mattress.

The mattress assembly, whether a single piece or more than one piece, defines a support surface. In case the mattress assembly is divided in a mattress and a mattress topper, preferably the support surface is defined on the mattress topper. The support surface is a surface where a body may lie for sleeping or resting activity. The body may be a human body or an animal body. The support surface is preferably a standard surface, that is, it may have the geometrical shape of a mattress surface. The support surface may include the upper surface of a mattress. Preferably, the support surface has a rectangular shape. Preferably, the dimensions of the support surface are those of a standard mattress surface, for example in case of an adult mattress, the width of the support surface may be comprised between 80 centimetres and 200 centimetres. The length of the support surface can be for example comprised between 190 centimetres and 220 centimetres. In other cases, the dimensions of the support surface are preferably: the width is comprised between 70 centimetres and 90 centimetres and the length is preferably comprised between 90 centimetres and 140 centimetres. The support surface may include more than one surface. For example, the support surface can be formed by the combination of two or more mattresses' top surfaces positioned close to each other. For example, two single mattresses may be located next to each other to form a double mattress and the support surface includes both top surfaces of the two single mattresses. Furthermore, the support surface does not need to match the complete top surface of the mattress. For example, two single mattresses may be located next to each other to form a double mattress and the support surface may include only one of the two top surfaces of the mattresses.

The mattress assembly includes a first array of gas sensors. Each gas sensor of the first array is adapted to measure the amount of a first gas, for example the concentration of the first gas, at a given location. The location where the amount of first gas is measured is a surrounding of a portion of the support surface. The support surface may be considered as divided in several portions, forming a first array of surface portions. The number of surface portions may depend on the size of the support surface. The number of surface portions may depend on the accuracy desired from the system. The number of surface portions may depend on the number of gas sensors. Preferably, to some or to all surface portions of the support surface a gas sensor of the first array is associated. The gas sensor is thus adapted to measure the amount of a first gas in the surrounding of the surface portion of the support surface is associated to. Therefore, the gas sensor is adapted to measure the concentration of the first gas in a surrounding of this portion of the support surface, meaning it measures the concentration of the first gas in a volume having as a side the portion of support surface. The number of surface portions into which the support surface is divided is preferably comprised between 25 and 1600 per square meter. Preferably, the surface portions of the support surface may have different areas one from the other. For example, wider portions may be present at the boundary of the support surface, while smaller portions may be present close to the centre of the support surface. In this way, a higher accuracy may be achieved where the probability of finding a body is the highest.

The first array of gas sensors may be distributed in an orderly configuration, with a constant pacing separating one sensor from the other. The first array of gas sensors may lie on a single plane. The first array of gas sensors may be located within the mattress assembly. Preferably, the first array of gas sensors may lie below the support surface.

Preferably, from the first array of gas sensors, it is possible to collect data from which a two dimensional (2D) map or a table of the concentration of the first gas at the support surface can be obtained. In the 2D map, to each or only to some surface portions of the first array of the support surface a value of the first gas concentration as measured by one of the gas sensors of the first array may be associated. In this way, the collected data are “position dependent”, because the value of the concentration is associated to the position where such a concentration has been measured. A resolution comprised between 2 centimetres and 15 centimetres is preferred, so that it is possible to determine a first gas concentration variation, if present, between two points located at a distance between 2 centimetres and 15 centimetres in the support surface.

The 2D map or table can be a time-varying map, due to the fact that the concentration of the first gas in one or more surface portions of the first array of the support surface may change. The concentration of the first gas in one or more surface portions of the first array of the support surface may change with time, and this change with time may take place only in one or more surface portions and not in others. For example, if the head of a person is positioned in a specific surface portion of the support surface, due to the person's breathing, the quantity of oxygen or of carbon dioxide may vary with time.

The 2D map may be used as a tool to determine the position of one or more body parts when a body is lying on the support surface. For example, from the 2D map, it can be determined that, over time, only in the surrounding of one or two surface portions of the first array of the support surface the oxygen concentration (first gas) in the air changes. For example, from the 2D map, it can be determined that, over time, only in the surrounding of one or two surface portions of the first array of the support surface the carbon dioxide (CO₂) concentration (first gas) in the air changes. It can be therefore determined that in that location, that is, in the location corresponding to the one or more surface portions of the first array where a change in the first gas is identified, the head of the person is present. If, for example, the hydrogen sulfide (H₂S) concentration in the ambient air varies in a single location, corresponding to a certain surface portion of the support surface, it can be determined that in that surface portion the nether part of the body may be located.

Checking the concentration of the first gas, and checking the variation of the concentration of the first gas with time, may allow to determine the position of one or more body parts. The variations in concentration of the first gas in a specific surface portion (or in more than one surface portion, but not in all surface portions) may indicate that in that specific surface portion a part of the body capable of changing the first gas concentration is present.

The system may include a control unit. The control unit is adapted to receive and elaborate data coming from the first array of gas sensors. The control unit may be comprised in the mattress assembly. The control unit may be external to the mattress assembly. The control unit may be responsible for generating the 2D concentration map or table. The control unit may be included in the external device to which data relative to the first gas amount as measured by the first array of gas sensors are sent.

The system also includes a transmitter adapted to send a signal containing data relative to the first gas concentration. The signal containing the data are received by an external device. The data may include information about all first gas concentrations as measured by all gas sensors in the first array. In this way for example, the external device may create the above mentioned 2D map or table. The data may contain information about the concentrations as measured by only some of the gas sensors in the first array. For example, only the maximum, or the minimum, first gas concentration is sent. The data may contain information about the concentration as measured by only one of the gas sensors in the first array. For example, only the first gas concentration measured by the gas sensor associated to the surface portion of the first array where a specific body part is located may be sent.

The control unit may also control the sampling frequency of the first array of gas sensors. The first array of the gas sensors may measure the concentration of the first gas at a given frequency. This frequency, called sampling frequency, may be variable and set by the control unit. The frequency at which the sampling takes place may be the same for all gas sensors of the first array, or it may be variable. The sampling frequency of a gas sensor may vary depending on the position of the gas sensor. The sampling frequency of a gas sensor may vary depending on the surface portion (and preferably the location within the support surface of this surface portion) the gas sensor is associated to. If the gas sensor is associated with a surface portion where the head is located, for example, the sampling frequency of that gas sensor may be higher than the sampling frequency of the gas sensor associated with the surface portion where the feet are located.

The control unit may elaborate the data coming from the first array of gas sensor. The control unit may determine in which surface portions there is a change in the first gas concentration with time. The control unit may determine in which surface portions a body part is present on the basis of the variations of the first gas concentration in these surface portions.

The signal may be sent after an elaboration. For example, the signal may be sent after an elaboration by the control unit. The elaboration may be a comparison with a threshold. The signal may be sent if the concentration of the first gas as detected by one of the gas sensors of the first array is above or below a certain threshold. The threshold is preferably a pre-set threshold and depends on the type of first gas the concentration of which is measured.

The control unit may include a memory unit and a central processing unit (CPU). The control unit may store and analyse data sent by the first array of gas sensors. The control unit may also associate the data to a particular user, so that to different users, different data are associated. Upon request of a user, the CPU may analyse the data and an evaluation report may be sent to the external device by the transmitter.

The signal may be sent by the transmitter only if one or more conditions are verified. Different conditions may be set. A condition may be for example a comparison with a threshold. For example, the signal is sent if the concentration of the first gas as measured by one or more of the gas sensors of the first array of the first gas is above or below a first threshold. Another condition may be a time comparison. The signal may be sent if the concentration of the first gas is above or below a threshold for longer than a pre-determined amount of time. Another condition may be the location where the first gas concentration is taken. For example, the signal is sent if the concentration of the first gas is coming from a gas sensor associated with a specific surface portion of the support surface. A combination of conditions may be used as well.

The signal which is sent may be not elaborated, and may contain only raw data of the measurements taken by the first array of gas sensors. The signal may contain the data relative to the measurements made by only one of the gas sensors of the first array, or may contain the data of the measurements made by a selection of gas sensors or by all gas sensors.

The data may be elaborated when received by the external device. For example, the comparison with the threshold, or a time, or the determination of the location from where the data are coming from, may be made at the external device.

The comparison with a threshold may be useful to detect potential risks for a person lying on the support surface. A comparison with more than one threshold may be made as well. A comparison may be made with a first threshold and a second threshold, the first threshold being different from the second threshold. For example, if the concentration of the first gas is above or below a first threshold, the signal is sent. If the concentration is above or below a second threshold, then the signal is sent only if such a concentration remains above or below the second threshold for at least a given time interval. For example, in case the first gas is CO₂, a first threshold may be set, which is for example equal to 13.000 parts per million. If the concentration of the CO₂ as measured by the first array of gas sensors is above the first threshold, a risk of suffocation may be present. An alerting signal may be sent by the system to the external device. This alerting signal may advise for attention on the person on the support surface. Already lower levels of CO₂ concentration (for example lower than a second threshold in the range of 12.000-13.000 parts per millions), if lasting for a certain amount of time, might lead to sleep disorder or a chronic insomnia. Thereby a signal could be sent to the external device, if the second threshold is exceeded for longer than the certain amount of time, warning of possible sleep disturbances of the person on the support surface. Another characteristic gas like carbon monoxide (CO) may also serve as an indicator of a suffocation incident or other health issues. A suffocation risk may also be present, when a person is suffering from nausea and vomiting. This vomit could block the airway of the person and may result in the suffocation risk. The system of the invention may detect, for example, the gastral fluid mix including lactic acid which when in contact with the atmosphere would form a characteristic gaseous mixture, for example a mixture mercaptan/sulfides (RSH). A signal may be sent if the RSH concentration as measured by one or more of the gas sensors of the first array is above a first threshold, for example if concentration of RSH >0.6 parts per million. Also, lower levels of RSH concentration (for example lower than a second threshold in the range of 0.1-0.5 parts per million), if this concentration lasts for a certain amount of time, might indicate gastrointestinal problems. Thereby a signal may be sent to the external device, for example to the phone of medical doctors, parents, family members or nursing staff, warning them of upcoming health issues, if the second threshold is exceeded for longer than the certain amount of time. The signal may also be used to wake up or alert the person on the support surface if a gas concentration is outside of the safe range. The measurements may be recorded or stored so that the user can review them later on, to self-assess whether there has been any data of gas concentration outside the selected ranges while the sleeper was laying on the support surface.

Additional physiological effects, like a bowel movement, may also be detected by the system of the invention. For example, the first array of sensors may be used to measure the hydrogen sulfide (H₂S) concentration in the ambient air. If the amount of hydrogen sulphide is lower than a first threshold, where the first threshold is for example equal to 2 parts per million, a signal is sent to the external device. An advice may be given, for example to control a diaper of the sleeper. Lower levels of H₂S concentration (for example lower than a second threshold in the range of 0.1-1.0 parts per million) might indicate gastrointestinal problems.

The threshold may also vary depending on the location of the surface portion associated to the measured first gas concentration. If it has been determined that in a specific surface portion a body part of interest is present, the threshold of the first gas concentration to which the gas concentration measured by the sensor associated to that specific surface portion is compared may be different than the threshold to which the gas concentration measured by the sensor associated to other surface portions is compared.

The data from the system may be sent to the external device at a certain frequency. Alternatively, the signal may be sent only when a condition is met. The condition can be the result of a comparison with a threshold. The frequency may be tunable.

The external device is preferably external to the system. The external device may be any type of device configured to operate or communicate with the system. Preferably, the external device is a device adapted to operate and communicate in a wireless environment. By way of example, the external device may be configured to transmit or receive wireless signals, and may include a user equipment, a mobile station, a fixed or mobile subscriber unit, a pager, a cellular telephone, a personal digital assistant (“PDA”), a Smartphone, an IPhone, a laptop, a netbook, a personal computer, a wireless sensor, consumer electronics, and other transmitter/receivers known in the art. The external device can be connected to a human or also to a machine.

The concentration of the first gas is measured over time. Preferably, the step of detecting the gas concentration and sending a signal representative of the concentration of the first gas to an external device are repeated. The repetition may take place at a given frequency. The repetition may take place during a time interval. Preferably, the time interval is pre-selected. This repetition of the steps of detecting and sending allow to verify whether variations in the first gas concentration are present over time. As mentioned, these variations may allow to determine in which surface portion of the support surface a body part is located.

The system of the invention is therefore very reliable in detecting gas concentrations in the surrounding of a support surface, where a body of a person can lie, due to the presence of a first array of sensors to measure the concentration in different surface portions of the support surface. In this way, potentially harmful conditions for the person on the support surface can be detected and the harm prevented. Furthermore, the measurement of the concentration of a first gas in different surface portions may be used to identify the location of different body parts on the support surface because different body parts may have a different effect on the first gas concentration in their surroundings. The measurement of the concentration of the first gas with time also allows to detect “localized” gas concentration variations, which in turn may allow to detect the position of various body parts.

Preferably, the system comprises one or more position sensors adapted to detect the position of a part of a body when located on the support surface. Preferably, the position sensors are integrated in the mattress assembly. The position sensors may determine the location of a body part, more preferably of more than one body part. The body parts are those of a body lying on the support surface. The detection of the position of one or more body parts may help in simplifying the elaboration of the signals coming from the first array of gas sensors because it is already known in which surface portions of the support surface changes in the first gas concentration may be expected. The body part may be for example the head of the body. The body part may be for example the nether region.

Preferably, the first array of gas sensors is adapted to measure an amount of the first gas in the surrounding of the position of the part of the body detected by the one or more position sensors. The localization of a specific body part may help in elaborating the signals coming from the first array of gas sensors. Already knowing where (that is, in which surface portion of the support surface) the changes in the first gas concentration may happen may help to minimize errors in the reading. It may also help in weighting in a different way the various signals coming from the first array of gas sensors, depending on the location of each gas sensor. For example, the measurements from a gas sensor associated to a surface portion of the support surface where the head of the body is present are more “important” than measurements coming from surface portions where other body parts are present. The accuracy and the responsiveness of the system may be improved.

Preferably, the first array of gas sensors is adapted to measure one or more of the following gasses: carbon monoxide, carbon dioxide, methanethiol, hydrogen sulphide. Preferably, the carbon dioxide, carbon monoxide, and methanethiol are measured in a surrounding of the head of a person. Preferably, the signal related to the first gas concentration coming from the first array of sensors is compared with a threshold that is:

| | |
|---|---|
| CO₂ concentration | The set threshold for the concentration is |
| | concentration above a threshold in the range |
| | between 12.000 parts per millions and 15.000 |
| | parts per millions |
| CO concentration | The set threshold for the concentration is |
| | concentrations below a threshold in the range |
| | between 100 parts per millions and 900 parts |
| | per millions |
| RSH threshold | The set threshold for the concentration is |
| | concentrations below a threshold in the range |
| | between 0.001 parts per millions and 0.9 parts |
| | per millions |
| H₂S threshold | The set threshold for the concentration is |
| concentration | concentrations within the range between |
| | 0.001 parts per millions and 1.9 parts per |
| | millions |

An alert signal may be sent from the system to the external device when one or more of the above conditions are met, that is, when the first gas concentration is above or below the threshold associated to the first gas. Alternatively, the signal is sent and the external device performs the comparison between the concentration as measured by the first set of sensors and the threshold associated to the first gas. The external device may generate the alert signal if the first gas concentration is above or below the threshold associated to the first gas. If a specific body part is detected, the system or the external device may weigh the signals coming from gas sensors located in proximity of the specific body part differently than the other signals.

Preferably, the mattress assembly includes: a cover layer defining the support surface. Preferably, the mattress assembly includes: a sensor layer including the first array of gas sensors, the sensor layer being located below the cover layer. The cover layer preferably is so designed to be “comfortable” for a human body when lying on it, due to the fact that it includes a support surface. Preferably, the cover layer is permeable to the first gas, so that the cover layer does not block the flow of the first gas through it. In this way, the first array of gas sensors can measure the first gas concentration, being located below the cover layer. Preferably, the cover layer has an air-porous foam structure. The cover layer may include thermoplastic polyurethane. The first gas may thereby reach the sensor layer by the high vapour permeability of the thermoplastic polyurethane. Preferably, the cover layer is flexible. Preferably, the cover layer has a thickness comprised between 2 millimetres and 15 millimetres, more preferably between 6 millimetres and 10 millimetres.

The sensor layer includes the first array of gas sensors. Preferably, the gas sensors in the first array are orderly spaced. Preferably, the sensor layer comprises a plurality sensor elements, each sensor element of the plurality including a gas sensor. The sensing elements are then attached to each other forming the sensor layer. The sensing elements may be identical in geometrical shape to each other. The sensing element may be attached to each other in a “tile structure”. Preferably, one or more spacing element may be interposed in between the sensing elements. Thus, the sensor layer may include sensor elements and spacing elements connected to each other so as to form a layer (the sensor layer). Preferably, the spacing elements have the same geometrical shape of the sensing elements, without the gas sensor. Preferably, the sensor elements or the spacing elements, or both, have a hexagonal structure. The sensor layer may thus have a resulting honeycomb structure.

The sensor layer may be a plastic layer, more preferably a flexible plastic layer. Preferably, the sensing element is formed in plastic. The sensor layer may include perforations. Preferably, the sensing element is formed in flexible plastic. “Flexible” means that the sensor layer preferably results “comfortable” to a person lying on it. It thus preferably accommodates to the weight of a body located on top of it. The sensing element includes at least one gas sensor, however it may include more than one gas sensor, depending on its size. The sensing element may include gas sensors of different types, for example gas sensors measuring concentrations of different gasses. Preferably, the gas sensor is formed on the sensing element by ink printing, sputtering or vapour disposition. The sensing element may comprise perforations, to save material and to permit eventually entering liquids to pass through the sensing element.

The spacing element may also be realized in plastic, more preferably in flexible plastic.

On a top view of the mattress assembly, each gas sensor of the first array is preferably associated to a surface portion of the support surface. The surface portion of the support surface may be a circle surrounding the gas sensor positioned underneath the surface portion in the top view. The support surface may thus be divided in a first array of surface portions, and to each surface portion a gas sensor located below the surface portion is associated.

The gas sensors in the first array may be electrically connected to each other. They may be electrically connected to each other in clusters. For example, the first array of gas sensors may be divided in gas sensors strips. The gas sensor strips may be positioned parallel to each other.

The sensor layer may include a first sensor layer and a second sensor layer. The first sensor layer includes the first array of gas sensors adapted to measure the concentration of the first gas. The second sensor layer includes a second array of gas sensors adapted to measure the concentration of a second gas. Preferably, the first layer of the first array of gas sensors and the second layer of the second array of gas sensors may be located one above the other. Preferably, the first gas is different from the second gas. Preferably, the gas sensors in the second array are orderly spaced. Preferably, the second sensor layer is divided in sensor elements as detailed for the first sensor layer. Thus, the second sensor layer may include sensor elements and spacing elements connected to each other so as to form the second sensor layer. Preferably, the spacing elements have the same geometrical shape of the sensing elements, without the gas sensor. Preferably, the sensor elements or the spacing elements, or both, have a hexagonal structure. The second sensor layer thus has a resulting honeycomb structure. When the first sensor layer and the second sensor layer are positioned one above the other, preferably the sensing elements of the first sensor layer correspond, in a top view, to the spacing elements of the second sensor layer.

The sensor layer may include, disposed on the same layer, the first array of gas sensors and a second array of gas sensors adapted to sense a second gas. Preferably, the first gas is different from the second gas. For example, the sensor layer may include alternating gas sensor of the first array and gas sensor of the second array.

Preferably, the sensor layer has a thickness comprised between 2 millimetres and 15 millimetres, more preferably between 5 millimetres and 10 millimetres.

Preferably, the system includes a sensor housing. The sensor housing preferably houses the sensor layer. For example, the sensor housing defines an inner sensing chamber having a ceiling and a bottom. Preferably, the sensor layer is attached to the ceiling of the sensor housing. In this way, a gap is present between the bottom of the sensor housing and the sensor layer. In case liquids enters the sensing chamber, they collect due to gravity to the bottom. Due to the fact that the sensor layer is attached to the ceiling, the sensors of the sensor layers may be separated from the liquid collected at the bottom. The sensor housing may be realized in polyethene. Preferably, the sensor housing is slightly elastic. Preferably, the sensor housing is stiff enough to create a resilient cavity (the sensing chamber) to include and protect the sensor layer adequately.

Preferably, the sensor housing has a thickness comprised between 8 millimetres and 30 millimetres, more preferably between 10 millimetres and 20 millimetres.

Preferably, the mattress assembly includes a gas-permeable layer interposed between the cover layer and the sensor layer. The gas permeable layer interposed between the cover layer and the sensor layer is gas permeable, so that the air from the surrounding of the support surface may pass through it and the first gas contained in the air may be detected by the sensor layers. The gas permeable layer may include a mesh structure. The mesh structure may be formed by polyethene yarns which create a resilient 3D structure with elastic properties. The gas permeable layer may include a pillar structure. The pillar structure may include a plurality of connected polyethene tubes or ducts. The gas permeable layer has preferably a stiffness that can support a pressure load of a body lying on the support surface.

Preferably, the gas permeable layer has a thickness comprised between 2 millimetres and 20 millimetres, more preferably between 8 millimetres and 12 millimetres.

Preferably, the transmitter is a wireless transmitter. The wireless transmitter is adapted to send data representative of the measurement performed by the first array or second array of gas sensors. The wireless transmitter preferably comprises an antenna to send the data. Preferably, the wireless transmitter sends an electromagnetic signal carrying information related to the first gas or second gas concentration.

Preferably, the system comprises a second array of gas sensor adapted to measure the amount of a second given gas in the surrounding of a second array of portions of the support surface and adapted to output data indicative of the measured amount of the second given gas. The first array of portions of the support surface and the second array of portions of the support surface may coincide. The first array or the second array of gas sensors may include metal oxide sensors and carbon nanotube sensors. Other sensors, not only gas sensors, may be comprised in the mattress assembly, for example biosensors or sensors responsive to temperature, liquid or sweat.

Preferably, the mattress assembly includes a mattress topper. Preferably, the mattress assembly also includes a mattress. Preferably, the mattress topper is fastened to the mattress. For example, the mattress topper may be fastened to the mattress using standard attachments used in the field. The mattress topper may include elastic retaining straps. The elastic straps may connect the mattress topper to every mattress corner of the mattress. The elastic retaining straps may hold the mattress topper in position by its stretching forces. The matrass topper may include hook-and-loop fasteners attaching the mattress topper to the mattress.

Preferably, the first array of gas sensors or the second array of gas sensors includes a plurality of strips of electrically connected gas sensors. In case a concentration 2D map is created, several concentration 2D maps can be realized, one per type of gas, for example a first 2D gas concentration map for the first gas and a second 2D gas concentration map for the second gas. The electrical connection preferably transports all signals coming from the first array of gas sensors or the second array of gas sensor to the control unit.

Preferably, the one or more position sensors comprises a temperature sensor. Preferably, the one or more position sensors comprises a weight sensor. Preferably, the one or more position sensors comprises a camera. Preferably, the one or more position sensors comprises a combination of two sensors among temperature sensor, camera and position sensor.

Preferably, the first array of gas sensors or the second array of gas sensors comprises: a common substrate. Preferably, the first array of gas sensors or the second array of gas sensors comprises: a plurality of gas sensors attached to the common substrate. Preferably, the substrate can be divided in the sensor elements and spacing elements. Preferably, the substrate includes perforations. Preferably, the substrate is flexible.

According to another aspect, the invention relates to a method to detect a first gas, the method comprising: providing a mattress assembly defining a support surface. The method may also comprise identifying in the support surface a first array of surface portions. The method may also comprise detecting an amount of a first gas present in a surrounding of each of the surface portion of the first array of surface portions. The method may also comprise sending to an external device a signal containing data indicative of the measured amount of the first gas.

The characteristics and advantages of the method of the invention have been already detailed with reference to the description of the system of the invention and are not herewith repeated.

The signal may contain data relative to a measurement coming from a single gas sensor of the first array or from a plurality of gas sensors of the first array. For example, the first array of gas sensors can be used to detect the position of a specific body part. This body part may be the head of a body. When the specific body part has been identified, that is, when the position of the body part has been identified, then only signals relative to data coming from the gas sensor(s) of the first array associated with the surface portion(s) of the support surface where the specific body part is located may be sent to the external surface.

Preferably, the method comprises positioning a body on the support surface. Preferably, the body is a human body.

Preferably, the method comprises: detecting the position of a part of the body positioned on the support surface. A single body part may be detected or more than a single body part, like two distinct, spatially separated, first body part and second body part, may be detected. The position of the body part may also indicate the orientation of a body part and not only its location. For example, the direction in which the head is located can be detected, because the concentration of the first gas, such as oxygen or carbon dioxide, is likely to change more frequently with every breath closer to the mouth than at backside of the head. Preferably, the matrass assembly includes a first array of gas sensors adapted to detect the amount of a first gas in a surrounding of the first body part and a second array of gas sensors adapted to detect the amount of a second gas in a surrounding of the second body part.

Preferably, the method comprises: determining in which surface portion of the first array of surface portions the detected part of the body is located. Preferably, only data coming from gas sensors associated to those portions of the support surface are sent to the external device. Preferably the sampling frequency of the gas sensors associated to those portions of the support surface is higher than the sampling frequency of the gas sensors associated to the remaining surface portions of the support surface containing other body parts or no body part at all.

Preferably, determining in which surface portion of the first array of surface portions the detected part of the body is located includes determining in which surface portion of the first array of surface portions the detected part of the body is located on the basis of the variations in the detected amount of first gas in that surface portion. Thus, the location of the body part is determined on the basis of detected variations in the amount (concentration) of gas in that specific surface portion. The variations are visible detecting the gas concentration over time.

Preferably, the step of sending a signal includes the step of sending a signal to a mobile device. The step of sending a signal preferably includes the step of sending a signal to a home automation system.

Preferably, the step of detecting an amount of a first gas comprises: forming a first array of gas sensors integrated in the mattress assembly. Preferably, the first array of gas sensors is an integral part of the matrass assembly and more preferably of the matrass topper.

Preferably, the step of forming a first array of gas sensors includes: ink printing, sputtering or depositing a suitable material to form the first array of gas sensors on a common substrate.

Preferably, the method comprises: forming a two-dimensional map of the concentration of the first gas in the surrounding of the support surface. Preferably, in the support surface a first array of surface portions can be identified. To each of those surface portions, a gas sensor of the first array of gas sensors is associated. Preferably, a map is created in which for each surface portion of the support surface, the concentration of the first gas as measured by the gas sensor of the first array associated to that surface portion is recorded. In this way, a map of the different concentrations of the first gas which is position dependent can be formed. The 2D map can be created either by the control unit, part of the system, or by the external device. Preferably, the two dimensional map may be visualized on a display. The display may be part of the external device.

Preferably, the method comprises forming a time dependent two-dimensional map of the concentration of the first gas in the surrounding of the support surface. The concentration of the first gas may vary with time. Therefore, the two dimensional map is updated with the new data coming from the first array of sensors. The update can take place at a given frequency. The two dimensional map may help to identify potential harmful situations if there are first gas concentration changes in the proximity of certain body parts. For example, an increasing concentration of CO₂, for example a concentration of CO₂ exceeding a first threshold, around the head of a person may trigger a warning alarm signal. The same concentration change in proximity of the feet of the body would not trigger such alarm signal.

In the present text, spatial coordinates are given with reference to a frame of reference in which a matrass assembly is positioned according to its standard use. This means that in such a frame of reference the support surface is a substantially horizontal surface. In turn then, the meaning of the terms “below”, “above”, refer to the configuration of the mattress assembly when in use. It is to be understood that the mattress assembly, when transported, assembled or stocked, may be rotated in space with respect to this configuration. A corresponding rotation of the frame of reference is thus to be made.

A “layer” in the present context defines a sheet of material having a thickness smaller than its other two dimensions by at least a factor of 10. Preferably, the other two dimensions are one order of magnitude larger than the thickness of the sheet.

“Flexible” in the present context denotes the ability of a layer to change shape without breaking when a force is applied.

In the present text, the verbs “comprise” and “include” are synonyms and they both indicate a non-exhaustive list of features. The verb “consist” indicates an exhaustive list.

The invention is defined in the claims. However, below there is provided a non-exhaustive list of non-limiting examples. Any one or more of the features of these examples may be combined with any one or more features of another example, embodiment, or aspect described therein.

Example Ex1: A system to detect a first gas, the system comprising:
a mattress assembly defining a support surface including:
   a first array of gas sensors, each gas sensor of the first array being adapted to measure an amount of a first gas in the surrounding of a portion of the support surface and adapted to output data indicative of the measured amount of the first gas, so that the amount of the first gas in a first array of surface portions of the support surface is sensed over time; and
   a transmitter adapted to send a signal to an external device containing data indicative of the measured amount of the first gas.

Example Ex2: The system as in Ex1, comprising one or more position sensors adapted to detect the position of a part of a body when located on the support surface.

Example Ex3: The system as in Ex2, wherein the first array of gas sensors is adapted to measure an amount of the first gas in the surrounding of the position of the part of the body detected by the one or more position sensor.

Example Ex4: The system according to one or more of Ex1-Ex3, wherein the first array of gas sensors is adapted to measure one or more of the following gasses: carbon monoxide, carbon dioxide, methanethiol, hydrogen sulphide.

Example Ex5: The system according to one or more of Ex1-Ex4, wherein the matrass assembly includes:
a cover layer defining the support surface; and
a sensor layer including the first array of gas sensors, the sensor layer being located below the cover layer.

Example Ex6: The system according to Ex5, wherein the mattress assembly includes a gas-permeable layer interposed between the cover layer and the sensor layer.

Example Ex7: The system according to one or more of Ex1-Ex6, wherein the transmitter is a wireless transmitter.

Example Ex8: The system according to one or more of Ex1-Ex7, comprising:
a second array of gas sensors adapted to measure the amount of a second given gas in the surrounding of a second array of surface portions of the support surface and adapted to output data indicative of the measured amount of the second given gas.

Example Ex9: The system according to one or more of Ex1-Ex8, wherein the mattress assembly includes a mattress topper.

Example Ex10: The system according to one or more of Ex1-Ex9, wherein the first array of gas sensors or the second array of gas sensors includes a plurality of strips of electrically connected gas sensors.

Example Ex11: The system according to Ex2, where the one or more position sensors comprises one or more of:
temperature sensor;
weight sensor;
camera.

Example Ex12: The system according to one or more of Ex1-Ex11, wherein the first array of gas sensors or the second array of gas sensors comprises:
a common substrate; and
a plurality of gas sensors attached to the common substrate.

Example Ex13: A method to detect a first gas, the method comprising:
providing a mattress assembly defining a support surface;
identifying in the support surface a first array of surface portions;
detecting an amount of a first gas present in a surrounding of each of the surface portion of the first array of surface portions; and
sending to an external device a signal containing data indicative of the measured amount of the first gas.

Example Ex14: The method according to Ex13, comprising:
repeating the steps of detecting an amount of a first gas present in a surrounding of each of the surface portion of the first array of surface portions; and sending to an external device a signal containing data indicative of the measured amount of the first gas for a time interval.

Example Ex15: The method according to Ex13 or Ex14, comprising:
repeating the steps of detecting an amount of a first gas present in a surrounding of each of the surface portion of the first array of surface portions; and sending to an external device a signal containing data indicative of the measured amount of the first gas at a given frequency.

Example Ex16: The method according to one or more of Ex13-Ex15, comprising:
determining variations of the detected amount of first gas over time.

Example Ex17: The method according to Ex16, comprising:
determining variations of the detected amount of first gas over time in each surface portion.

Example Ex18: The method according one or more of Ex13-Ex17, comprising:
positioning a body on the support surface;

Example Ex19: The method according to Ex18, comprising:
detecting the position of a part of the body positioned on the support surface.

Example Ex20: The method according to Ex19 comprising:
determining in which surface portion of the first array of surface portions the detected part of the body is located.

Example Ex21: The method according to Ex20, wherein determining in which surface portion of the first array of surface portions the detected part of the body is located includes
determining in which surface portion of the first array of surface portions the detected part of the body is located on the basis of the variations in the detected amount of first gas in that surface portion.

Example Ex22: The method according to one or more of Ex13-Ex21, wherein the step of sending a signal includes the step of sending a signal to a mobile device.

Example Ex23: The method according to one or more of Ex13-Ex22, wherein the step of detecting an amount of a first gas comprises:
forming a first array of gas sensors integrated in the mattress assembly.

Example Ex24: The method according to Ex23, wherein the step of forming a first array of gas sensors includes:
ink printing, sputtering or depositing a suitable material to form the first array of gas sensors on a common substrate.

Example Ex25: The method according to one or more of Ex13-Ex24, comprising:
forming a two-dimensional map of the concentration of the first gas in the surrounding of the support surface.

Example Ex26: The method according to Ex25, comprising:
forming a time dependent two-dimensional map of the concentration of the first gas in the surrounding of the support surface.

Examples will now be further described with reference to the figures in which:

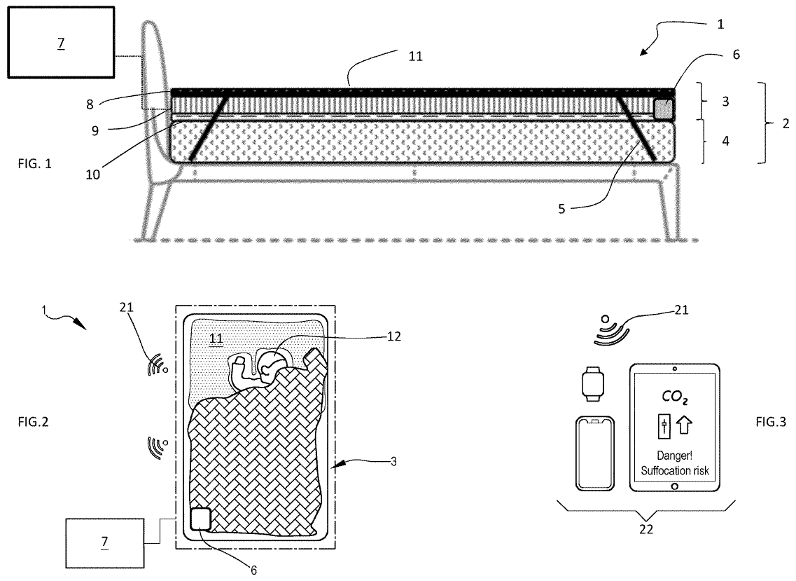

FIG. 1 is a side view of a system to measure the amount of a first gas including mattress assembly realized according to an embodiment of the invention;

FIG. 2 is a top view of the system of FIG. 1 in use;

FIG. 3 is a schematic view of different embodiments of external devices in communication with the system of FIGS. 1 and 2;

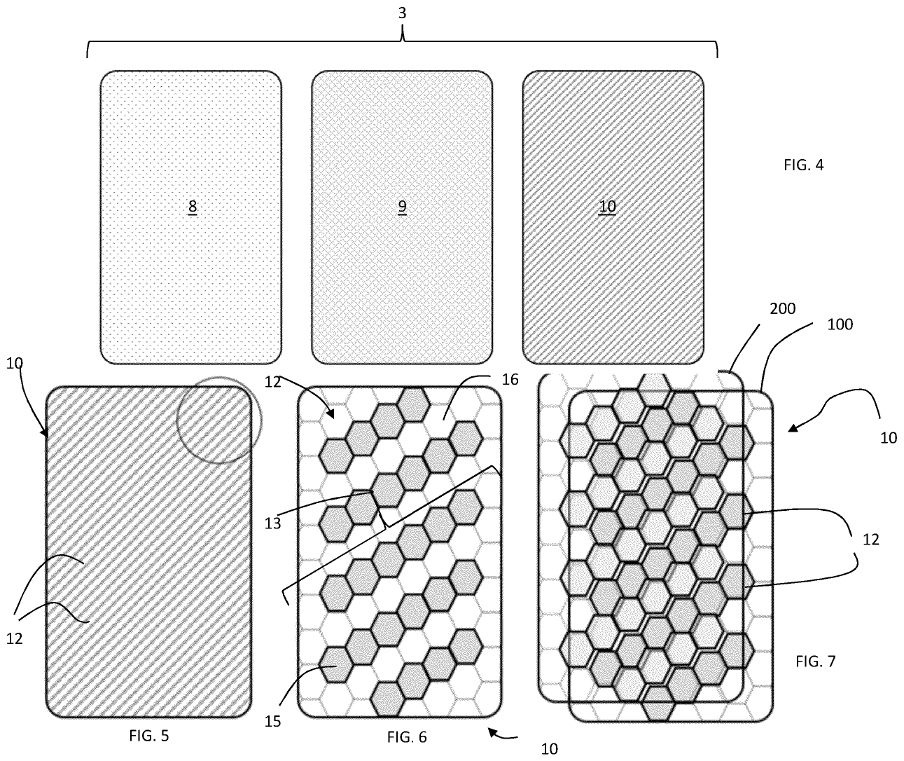

FIG. 4 is a top view of the mattress assembly of FIG. 1 in a disassembled configuration;

FIG. 5 is a top view of one of the layers of the mattress assembly of FIG. 4;

FIG. 6 is an enlarged top view of a detail of the layer of FIG. 5;

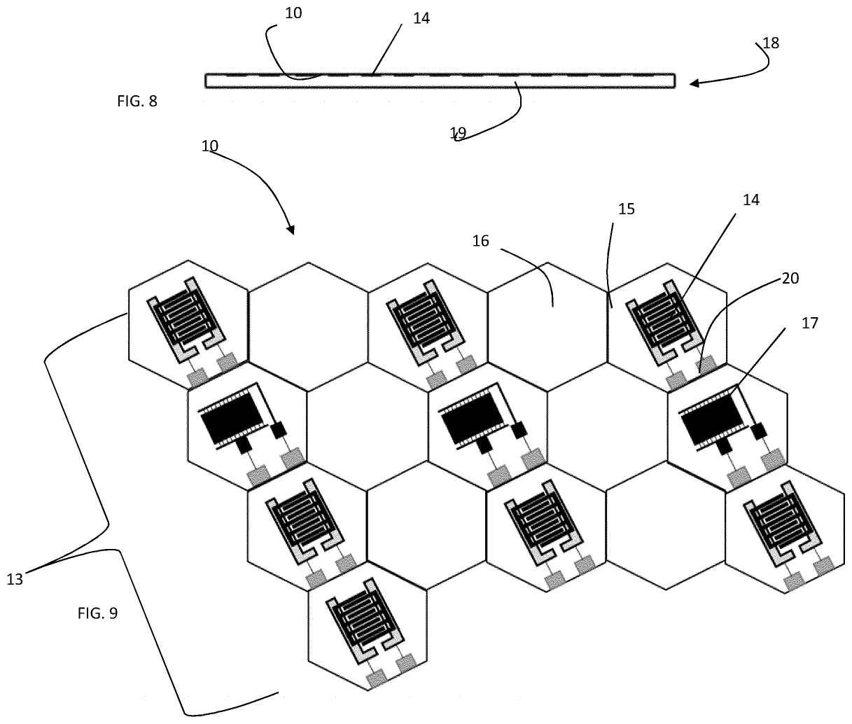

FIG. 7 is a top view of the detail of FIG. 6 in a disassembled configuration;

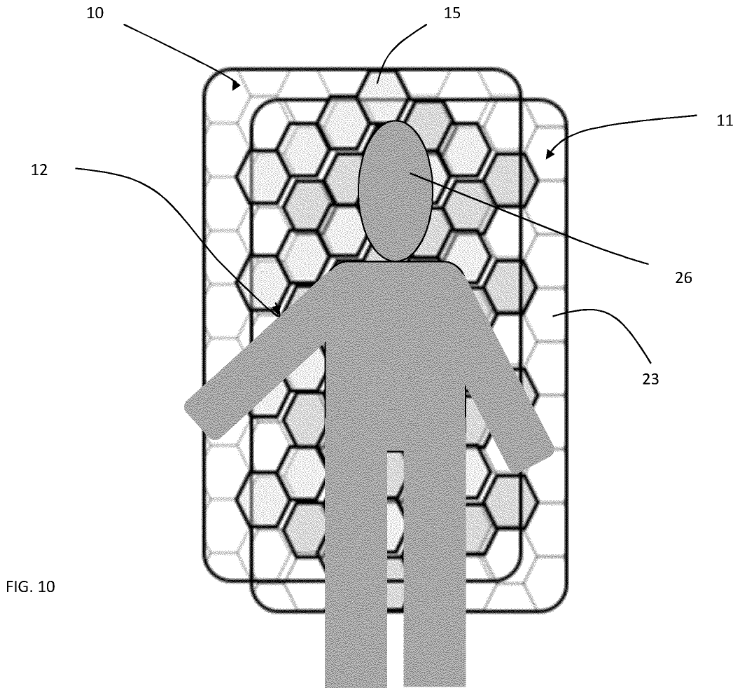

FIG. 8 is a side view of the layer of FIG. 5 in a different embodiment of the invention;

FIG. 9 is an enlarged top view of a detail of FIG. 6 or 7; and

FIG. 10 is a more detailed view of FIG. 2.

With initial reference to FIGS. 1 and 2, with 1 a system to measure an amount of a first gas is globally indicated with 1.

The system 1 includes a mattress assembly 2. The mattress assembly 2 includes a mattress topper 3 and a mattress 4. The mattress topper 3 is attached to the mattress 4 by means of elastic retaining straps, all indicated with 5, an elastic restraining straps 5 on every corner of the mattress 4. The retaining straps hold the mattress topper 3 in position by their stretching forces.

Furthermore, the system 1 includes, located within the mattress topper 3, a wireless transmitter 6 (sketched as a rectangle in the drawings 1 and 2).

The system 1 includes also a control unit 7, in communication with the transmitter 6.

The mattress topper 3 comprises a three-layered structure comprising a cover layer 8, a gas permeable layer 9 and a sensor layer 10. The three layers are depicted in an assembled configuration in FIG. 1 and separated one from the others in FIG. 4.

The cover layer 8 includes an air-porous foam structure and preferably is formed by thermoplastic polyurethane. The cover layer 8 is the upmost layer of the mattress assembly and defines a support surface 11 where a body 12 may lie. The gas present in the air in the surrounding of the mattress topper 3 can enter the cover layer 8 due to high vapour permeability of the thermoplastic polyurethane. In FIG. 2, the support surface 11 has the same dimensions and shape of a standard mattress upper surface. The thickness of the cover layer 8 is preferably comprised between 6 millimetres and 10 millimetres.

The gas permeable layer 9 is located below the cover layer 8 and is the middle layer of the mattress topper 3. The gas permeable layer 9 preferably has a permeable mesh structure. Preferably, the mesh structure of the air gas permeable layer 9 includes polyethene yarns which create a resilient 3D structure with elastic properties. Preferably, the thickness of the gas permeable layer 9 is of 10 millimetres.

The sensor layer 10 is the lowermost layer of the mattress topper 3, and it is preferably in contact to the mattress 3. With now reference to FIGS. 5-9, the sensor layer 10 includes a first array of gas sensors 12 which are preferably divided in sensor strips 13. The first array of gas sensors is adapted to measure the concentration of a first gas. The first array 12 of gas sensors shown in FIG. 5 is better detailed in the enlarged view of FIG. 6, where a small portion of the sensor layer 10 is depicted. In FIG. 6, the sensor strips 13 are visible. Preferably, the sensor strips 13 run parallel to each other and diagonally with respect to the sides of the sensor layer 10. Each sensor strip 13 comprises a plurality of gas sensors 14 (visible in FIG. 9) adapted to measure the concentration of the first gas. In order to form the sensor strips 13, preferably each sensor strip 13 comprises various modular sensor elements, such as sensor tiles 15, which are linked to each other by at least one side of the tile 15. Thus, the sensor strip 13 is formed by at least two sensor tiles 15 which are linked together to build the sensor strip 13. Preferably, the sensor strip 13 is formed by a plurality of sensor tiles 15 all having the same geometrical shape. Each sensor tile 15 includes at least a gas sensor 14 as shown in FIG. 9. The sensor layer 10 may include not only sensor tiles 15, but also preferably spacer tiles 16. The spacer tiles 16 enforce a distance between sensor tiles 15 belonging to different sensor strips 13. Several sensor strips 13 are preferably combined with spacer tiles 16, which connects at least two sensor tiles 15 belonging to two different sensor strips 13. Preferably, the sensor layer 10 is formed by sensor tiles 15, each of the sensor tiles including a sensor 14, and spacer tiles 16 without sensors. The sensor layer 10 is thus divided in strips, parallel to each other, which can be either sensor strips 13 or strips without sensors formed by spacer tiles 16. This configuration is clearly shown in FIGS. 6 and 9.

Preferably, sensor tiles 15 and spacer tiles 16 are identical in their geometrical shape and dimensions. Preferably, sensor tiles 15 and spacer tiles 16 have a honeycomb shape.

Preferably, the spacer tiles 16 and the sensor tiles 15 are made of a flexible plastic substrate and can comprise perforations (not visible in the drawings), to save material and to permit eventually entering liquids to pass through them.

As depicted in FIG. 7, the sensor layer 10 may comprise a first and second layer 100, 200, the first sensor layer 100 including the first array of sensors 12 to measure a concentration of the first gas, and the second sensor layer 200 comprising a second array of gas sensors (not shown) to measure the concentration of a second gas. The two layers 100, 200 may be combined one on top of each other. The first sensor layer 100 and the second sensor layer 200 both preferably include sensor tiles 15 and spacer tiles 16 as described above. Preferably, when the first sensor layer 100 and the second sensor layer 200 are one on top of the other, sensor tiles 15 of the second sensor layer 200 may be located above the spacer tiles 16 of the first sensor layer 100, and vice versa (see FIG. 7, where the two layers 100, 200 are slightly shifted to show the tiles structure of both of them that otherwise would coincide).

In a further embodiment depicted in FIG. 8, the sensor layer 10 can be arranged inside a sensor housing 18. The sensor housing 18 defines an inner chamber 19. As shown in FIG. 8, the sensor layer 10 is thereby attached to the upper part of the sensor housing 18, that is, at the ceiling of the inner chamber 19 in order to avoid that eventually entering liquids would accumulated on top of the sensor layer 10.

Preferably, the sensor housing 18 comprises polyethene walls having elastic properties and enough stiffness to create the inner chamber 19 to include and protect the sensor layer 10 adequately. Preferably, the thickness of the sensor housing 18 is comprised between 10 millimetres and 20 millimetres.

As illustrated in FIG. 9, each sensor tile 15 includes at least one gas sensor 14. Preferably, the gas sensor is flat and flexible. The gas sensor 14 is generally applied to the sensor tile 15 by ink printing, sputtering or vapour disposition. Several gas sensors 14, 17 measuring different gasses may be applied to the sensor tile 15 depending on the given amount of space on the sensor tile 15. For example, the tile sensor 15 may include a gas sensor 15 of the first array and a second gas sensor 17 of the second array, adapted to measure a first gas concentration and a second gas concentration, respectively. In a preferred embodiment, metal oxide sensors and carbon nanotube sensors are applied to the sensor tiles 15, whereby each sensor can be responsible for specific gas and a corresponding threshold level. In FIG. 9, each sensor tile 15 includes a single gas sensor, either a gas sensor 14 of the first array or a gas sensor 17 of a second array.

Other sensors like biosensors or sensors responsive to temperature, liquid or sweat (not depicted in the drawings) may be incorporated to the tiles.

All gas sensors 14, 17 are electrically interconnected by, for example, an OR-circuit with a contact area 20. The signals coming from the gas sensors 14, 17 are directed to the transmitter 6, which may include a communication module (not visible in the drawings), for example a PAN, LAN or WAN interface. The transmitter 6 may be controlled by the control unit 7. The transmitter 6 is adapted to send a signal 21, schematically depicted with a wave in FIGS. 2 and 3, to a connected smart device 22. In FIG. 3, several different smart devices 22 are shown, such as a smartwatch, a tablet or a smartphone. A single smart device 22 is needed, however more than one smart device can be used.

The functioning of the system 1 of the invention is as follow.

As depicted in FIG. 10, the support surface 11 may be divided in a first array of surface portions 23. Each surface portion 23 may have the shape of a sensor tile 15 or spacer tile 16, for example they have a hexagonal shape. It may however have any shape. To each surface portion 23, a gas sensor 14, 17 is associated. Therefore, each gas sensor 14, 17 of the first array or second array takes measurements of the concentration of the first gas or of the second gas in the surrounding of each surface portion 23 forming the support surface 11.

The gas sensors 14, 17, as controlled by the control unit 7, send signals regarding the concentration of the first gas or second gas in the surrounding of each surface portion 23 to the transmitter 6 or control unit 7. This sending is repeated over time. This concentration of the first gas or second gas may differ depending on the surface portion 23 from which the measurements are coming from. Therefore, the position of certain body parts of a body 12 on the support surface 11, for example of the head 26, may be detected. This is due to the fact that the concentration of certain gases may change only in the neighbourhood of certain body parts. These variations in concentration are monitored over time. Thus the position and even the orientation of body parts can be detected.

If the gas concentration of the first gas or second gas as measured by gas sensors 14, 17, in its variation over time, exceeds or is a certain threshold below in one or more surface portions 23, for example in the portions 23 where the head 26 is located, the signal 21 would be sent by transmitter 6 to the smart device 22, for example warning that an infant located on the support surface 11 suffers a suffocation risk from blanket covering the infant's face. This detection may take place as follow. Initially, the infant (body 12) at first would normally be breathing; however, the amount of CO₂ under the blanket would increase over time due to exhalation. The gas sensors 14, 17 in this situation may detect this continuous increase of CO₂, in particular where the head 26 is located, and when the detected concentration in the location where the head is crosses a threshold concentration of CO₂ (for example a concentration higher than 13.000 parts per million), the transmitter 6 sends an alerting signal 21 to a connected smart device 22 advising the parents for attention on the child. Also without a blanket covering the infant's face, already lower levels of CO₂ (for example comprised between 12.000 parts per million and 13.000 parts per million), which lasts over a certain amount of time, might lead to sleep disorder or a chronic insomnia. Thereby a message 21 could be send to the smart device 22, warning of possible sleep disturbances of the child.

Other characteristic gases like carbon monoxide (CO) could also serve as an indicator of a suffocation incident or other health issues.

The second array of sensors 17 could be used to detect, for example, the gastral fluid mix including lactic acid which, when in contact with the atmosphere, gives the characteristic gaseous mixture, that is the mixture mercaptan/sulfides (RSH). If the concentration of this mixture, as detected by the second array of gas sensors 17, is above a threshold level, for example it is above 0.6 parts per million, a signal 21 may be sent to the smart device 22. Also lower levels of RSH (for example a concentration of RSH comprised between 0.1 parts per million and 0.5 parts per millions), over a certain amount of time, might indicate gastrointestinal problems. Thereby a signal 21 could be send to the smart device 22 signalling a possible upcoming health issues.

Additional physiological effects, like a bowel movement, could also be detected by monitoring the hydrogen sulfide (H₂S) concentration in the ambient air. When detecting the occurrence of H₂S (lower than 2 parts per millions), a signal would be sent to the smart device 22 of a user with the advice to change the diaper of the infant or of an elderly. Lower levels of H₂S (for example comprised between 0.1 parts per million and 1.0 parts per millions) might indicate gastrointestinal problems.

The control unit 7 preferably includes a memory unit and a CPU (not shown in the drawings). Preferably the control unit 7 stores and analyses the data coming from the gas sensors 14, 17 and may monitor data of a specific user, for example different gas concentrations, temperature distributions or detected sweat on the mattress topper. Upon request of an user, the CPU could analyse the monitoring data and an evaluation report could be send to a user's smart device 22 by the communication module 6.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number A is understood as A ±10 percent of A. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed invention. Also, all ranges include the maximum and minimum points disclosed and include any intermediate ranges therein, which may or may not be specifically enumerated herein.

1-15. (canceled)

16. A system to detect a first gas, the system comprising:
a mattress assembly defining a support surface including:
a first array of gas sensors, each gas sensor of the first array being adapted to measure an amount of a first gas in the surrounding of a portion of the support surface and adapted to output data indicative of the measured amount of the first gas at a given frequency, so that the amount of the first gas in a first array of surface portions of the support surface is sensed over time; and
a transmitter adapted to send a signal to an external device containing data indicative of the measured amount of the first gas.

17. The system of claim 16, comprising a control until adapted to receive and elaborate data coming from the first array of gas sensors and configured for generating a 2D concentration map or table of the concentration of the first gas at the support surface.

18. The system of claim 16, comprising one or more position sensors adapted to detect the position of a part of a body when located on the support surface.

19. The system according to claim 18, wherein the first array of gas sensor is adapted to measure an amount of the first gas in the surrounding of the position of the part of the body detected by the one or more position sensor.

20. The system according to claim 16, wherein the first array of gas sensors is adapted to measure one or more of the following gasses: carbon monoxide, carbon dioxide, methanethiol, hydrogen sulphide.

21. The system according to claim 16, wherein the matrass assembly includes:
a cover layer defining the support surface;
a sensor layer including the first array of gas sensors, the sensor layer being located below the cover layer.

22. The system according to claim 16, wherein the first array of gas sensors includes a plurality of strips of electrically connected gas sensors.

23. The system according to claim 17, where the one or more position sensors comprises one or more of:
temperature sensor;
weight sensor;
camera.

24. The system according to claim 16, wherein the first array of gas sensors comprises:
a common substrate;
a plurality of gas sensors attached to the common substrate.

25. A method to detect a first gas, the method comprising:
providing a mattress assembly defining a support surface;
identifying in the support surface a first array of surface portions;
detecting an amount of a first gas present in a surrounding of each of the surface portion of the first array of surface portions; and
sending to an external device a signal containing data indicative of the measured amount of the first gas;
repeating the steps of detecting an amount of a first gas present in a surrounding of each of the surface portion of the first array of surface portions; and sending to an external device a signal containing data indicative of the measured amount of the first gas at a given frequency.

26. The method according to claim 25, comprising:
positioning a body on the support surface.

27. The method according to claim 26, comprising:
detecting the position of a part of the body positioned on the support surface.

28. The method according to claim 27 comprising:
determining in which surface portion of the first array of surface portions the detected part of the body is located.

29. The method according to claim 25, wherein the step of detecting an amount of a first gas comprises:
forming a first array of gas sensors integrated in the mattress assembly.

30. The method according to claim 29, wherein the step of forming a first array of gas sensors includes:
ink printing, sputtering or depositing a suitable material to form the first array of gas sensors on a common substrate.

31. The method according to claim 25, comprising:
forming a two-dimensional map of the concentration of the first gas in the surrounding of the support surface.
